(19) United States
(12) Patent Application Publication
(10) Pub. No.: US 2023/0173223 A1
(43) Pub. Date: Jun. 8, 2023

(54) URINARY CATHETER FOR ENDOSCOPE

(71) Applicant: Saint Louis University, St. Louis, MO (US)

(72) Inventor: Sameer Siddiqui, St. Louis, MO (US)

(73) Assignee: Saint Louis University, St. Louis, MO (US)

(21) Appl. No.: 18/061,047

(22) Filed: Dec. 2, 2022

Related U.S. Application Data
(60) Provisional application No. 63/264,815, filed on Dec. 2, 2021.

(51) Int. Cl. A61M 25/00; A61M 25/10; A61B 1/05; A61B 1/307

(52) U.S. Cl. CPC A61M 25/0017; A61M 25/0067; A61M 25/10; A61B 1/05; A61B 1/307

(57) ABSTRACT

The present disclosure relates to catheters, and more particularly, to urinary catheters that can be used with an endoscope as well as a catheter system comprising a catheter and an endoscope, wherein the endoscope is disposed in the lumen of the catheter. In one instance, a catheter system includes an endoscope and a catheter. The endoscope has an insertion tube sized and shaped to be inserted into a body lumen of a patient. The catheter includes an elongate body having an axis and proximal and distal end portions space apart from one another along the axis. The elongate body is sized and shaped to be received in the body lumen. The elongate body defines a lumen extending between the proximal and distal end portions. The lumen is sized and shaped to receive the insertion tube of the endoscope.

### CROSS-REFERENCE TO RELATED APPLICATION

This application claims priority to U.S. Provisional App. No. 63/264,815, filed Dec. 2, 2021, the entirety of which is hereby incorporated by reference.

### FIELD

The present disclosure relates to catheters, and more particularly, to urinary catheters that can be used with an endoscope as well as a catheter system comprising a catheter and an endoscope, wherein the endoscope is disposed in the lumen of the catheter.

### BACKGROUND

Urinary catheters are inserted into a person's bladder to allow urine to drain from the bladder and be collected, generally in a bag. One method of installing a urinary catheter generally involves inserting an endoscope and a guide wire, such as a cystoscope, into the bladder. The cystoscope is then withdrawn from the bladder and the urinary catheter is slide along the guide wire into the bladder. After the urinary catheter is positioned, the guide wire is withdrawn from the bladder.

Conventionally, a urologist is required for the insertion of a cystoscope. However, a urinary catheter can be installed by other healthcare professionals. Thus, there remains a need for a catheter with enhanced functionality that can be installed without the need for a cystoscope or urologist.

### SUMMARY

In one aspect, a catheter system comprises an endoscope and a catheter. The endoscope has an insertion tube sized and shaped to be inserted into a body lumen of a patient. The catheter includes an elongate body having an axis and proximal and distal end portions space apart from one another along the axis. The elongate body is sized and shaped to be received in the body lumen. The elongate body defines a lumen extending between the proximal and distal end portions. The lumen is sized and shaped to receive the insertion tube of the endoscope.

In another aspect, a method of using a catheter comprises inserting the catheter into a body lumen of a patient using an endoscope received in the catheter. The catheter comprises an elongate body having an axis and proximal and distal end portions space apart from one another along the axis. The elongate body defines a lumen extending between the proximal and distal end portions. The endoscope includes an insertion tube and the insertion tube is disposed in the lumen of the catheter.

Other objects and features of the present disclosure will be in part apparent and in part pointed out hereinafter.

### BRIEF DESCRIPTION OF THE DRAWINGS

FIG. 1 is a perspective of a catheter system according to one embodiment of the present disclosure.

FIG. 2 is a perspective of a catheter of the catheter system mounted on an endoscope of the catheter system.

FIG. 3 is a cross section of the distal end portion of the catheter mounting on the endoscope.

Corresponding reference characters indicate corresponding parts throughout the drawings.

### DETAILED DESCRIPTION

In general, the present invention relates to a catheter and a catheter system, and methods of using the catheter and catheter system. Unlike a cystoscope, the catheter and catheter system is designed for placement without a urologist. Further, the catheter and catheter system can be constructed for a single use (i.e., disposable), which eliminates the need for disinfection/sterilization cycles after use.

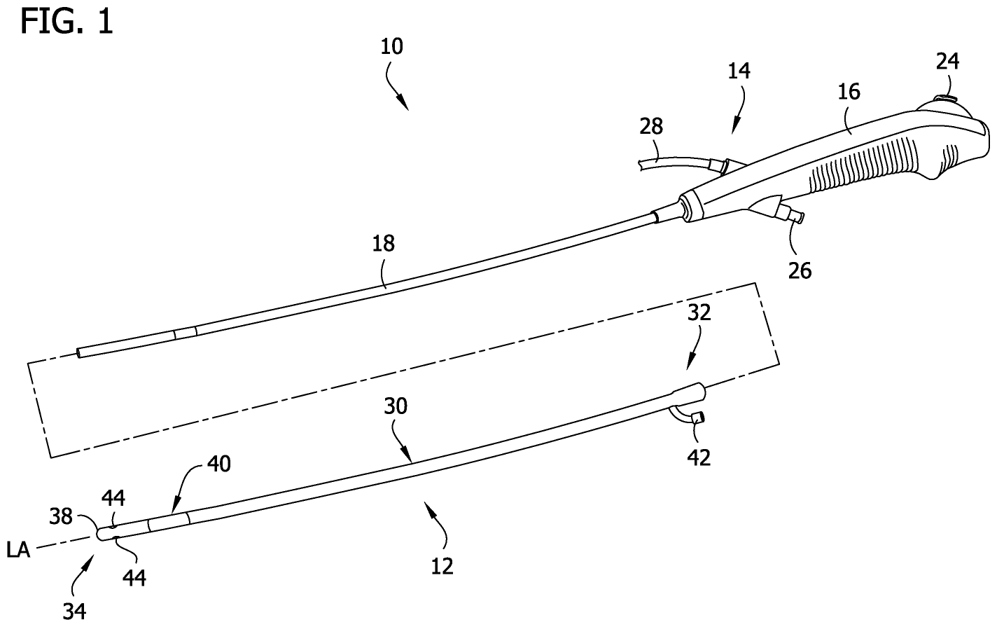

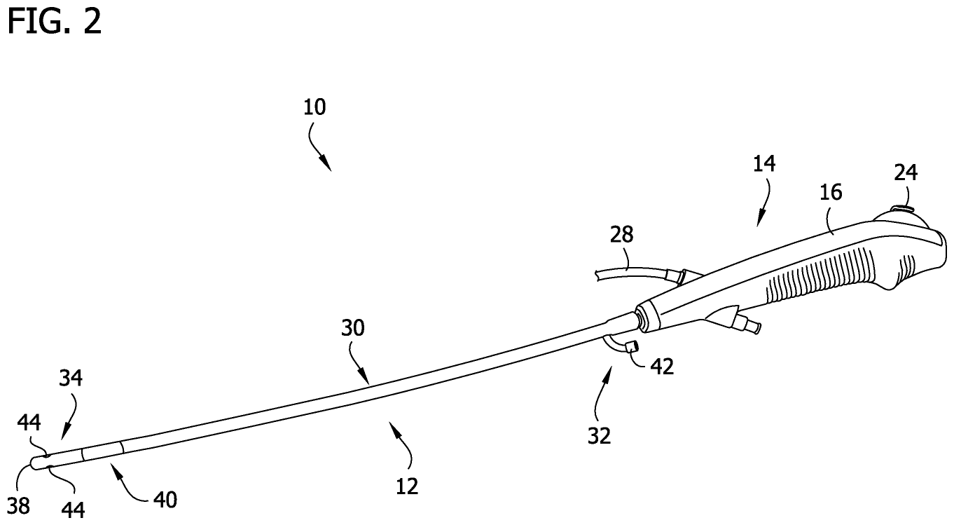

Referring to FIGS. 1-3, a catheter system for inserting a catheter into a body lumen of a patient or subject is generally indicated at reference number 10. The catheter system 10 includes a catheter 12 and an endoscope 14 (e.g., comprising a light source and camera). The illustrated catheter 12 is a urinary catheter suitable for inserting into the urethra of a patient to empty the patient's bladder, although other types of catheters are within the scope of the present disclosure. Similarly, the illustrated endoscope 14 can be an endoscope suitable for viewing the urethra and/or bladder of the patient, although other types of endoscopes (e.g., ureteroscope) are within the scope of the present disclosure. The catheter 12 is mountable on the endoscope 14 so that the endoscope can be used to insert the catheter into and along the body lumen of the patient.

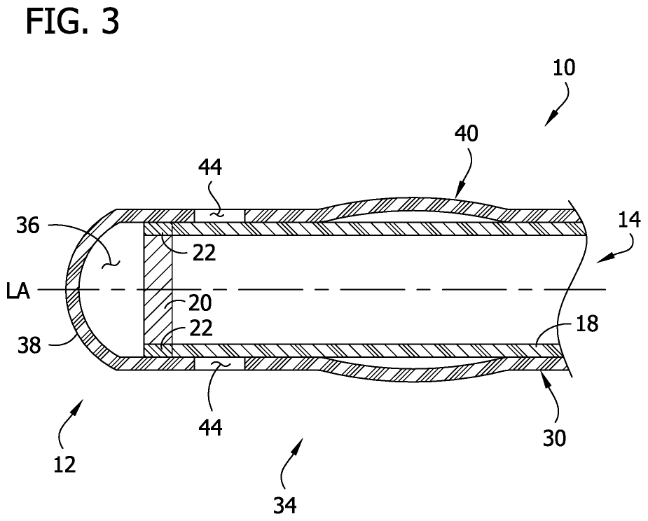

The endoscope 14 comprises a handle 16 and an insertion tube or cord 18 extending distally form the handle. The insertion tube 18 is sized and shaped to be inserted into the body lumen of the patient. The insertion tube 18 includes a camera 20 (FIG. 3) at the distal end of the insertion tube for viewing a portion (e.g., urethra, bladder, etc.) of the patient. The insertion tube 18 also includes one or more light sources 22, such as light emitting diodes (LEDs), at the distal end of the insertion tube for illuminating the portion of the patient. The handle 16 may include an actuator 24 operatively coupled to the insertion tube 18 for deflecting or bending at least a portion of the insertion tube. This helps guide the insertion tube 18 when the insertion tube is being inserted into and moved along the body lumen. The handle 16 may include an accessory port 26 (FIG. 1) for receiving and/or connecting to accessories used with the endoscope 26. The endoscope 14 includes a cable 28 (e.g., electrical cable) extending from the handle 16. The cable 28 can be connected to a display (not shown), such as a television or monitor, for viewing the image and/or video captured by the camera 20. In one embodiment, the endoscope 14 is a single-use, disposable endoscope. Other configurations of the endoscope are within the scope of the present disclosure.

The catheter 12 comprises an elongate body 30 having a longitudinal axis LA and proximal and distal end portions 32 and 34, respectively, spaced apart along the axis. The elongate body 30 is sized and shaped to be received in (e.g., for insertion into) a body lumen of a patient. The elongate body 30 defines a lumen 36 (e.g., insertion tube lumen or endoscope lumen). The lumen 36 extends between the proximal and distal end portions 32, 34 of the elongate body. The lumen 36 is sized and shaped to receive the insertion tube 18 of the endoscope 14. In one embodiment, the lumen 36 has a lumen diameter that is larger than an insertion tube diameter of the insertion tube 18 of the endoscope 14. In one embodiment, the lumen diameter of the lumen 36 is about 5.4 mm (about 0.21 inches). The lumen 36 can slidably receive the insertion tube 18 therein so that the catheter 12 can be mounted on the endoscope 14 and then advance through the body lumen by the endoscope. In one embodiment, the lumen 36 may have a lubricious inner surface for sliding the insertion tube 18 within the lumen. The lubricous surface may be provided by a lubricous polymer layer or a lubricious coating. The lumen 36 includes an open proximal end and an opposite closed distal end spaced apart along the longitudinal axis LA. The open proximal end of the lumen 36 is at the proximal end portion 32 and the closed distal end of the lumen is at the distal end portion 34. The open proximal end of the lumen 36 is sized and shaped to receive the insertion tube 18 of the endoscope 14 and the closed distal end prevents the insertion tube 18 of the endoscope 14 from being extend out of the lumen 36.

Referring to FIG. 3, the elongate body 30 of the catheter 12 includes a tip 38 at the distal end portion 34. The tip 38 defines the distal end of the elongate body 30. The tip 38 also defines the closed distal end of the lumen 36. In one embodiment, the tip 38 is transparent. This allows the endoscope 14 to view the portion of the patient's body by looking through the tip 38 of the catheter 12. The tip 38 may comprise (e.g., be made of) a transparent or first material, and the remainder of the elongate body 30 may comprise the same material as the tip or one or more different materials, or a combination thereof. For example, the tip 38 and the remainder of the elongate body 38 can be made of the same transparent material and therefore both be transparent. In another example, the tip 38 is made of a transparent material and the remainder of the elongate body 30 is made of one or more opaque materials.

With the catheter 12 mounted on the endoscope 14, the camera 20 of the endoscope is axially aligned with the transparent material of the tip 38. In one embodiment, the transparent tip 38 (e.g., transparent material) defines the entire closed distal end of the lumen 36. In one embodiment, the transparent tip 38 (e.g., perimeter of the transparent material) is directly connected to the side or cylindrical wall of the elongate body 30. In one embodiment, the transparent tip 38 (e.g., transparent material) defines the entire distal end of the elongate body 30. In one embodiment, the outward radial extent of the transparent material relative to the longitudinal axis LA is equal to or larger than the outward radial extent of the camera 20 relative to the longitudinal axis. In one embodiment, the outward radial extent of the transparent material relative to the longitudinal axis LA is equal to or larger than the outward radial extent of at least one (include all) the light sources 22.

The elongate body 30 of the catheter 12 may include an inflatable balloon 40 at the distal end portion 34. The inflatable balloon 40 is used to secure the catheter 12 in the body lumen. By inflating the inflatable balloon 40, the outer diameter of the inflatable balloon increases to inhibit the catheter 12 from unintentionally withdrawing from the body lumen. The catheter 12 includes an inflation port 42 fluidly coupled to the inflatable balloon 40 via an inflation lumen (not shown). The inflation port 42 is configured to be connected to an inflator (not shown) which delivers a supply of inflation fluid, such as water or air, to the inflatable balloon 40 to inflate the inflatable balloon.

The elongate body 30 defines at least one fluid inlet 44 in fluid communication with the lumen 36. In the illustrated embodiment, the elongate body 30 defines two (e.g., first and second) fluid inlets 44, although other amounts of fluid inlets are within the scope of the present disclosure. The two fluid inlets 44 are disposed on generally opposite sides of the elongate body 30. The two fluid inlets 44 are directly opposite one another. The fluid inlets 44 are disposed at the distal end portion 34. In the illustrated embodiment, the fluid inlets 44 are disposed proximally of the tip 38. In particular, the fluid inlets 44 are disposed between the tip 38 and the inflatable balloon 40. The fluid inlets 44 permit fluid to flow into the lumen 36 of the catheter 12.

The catheter 12 and the endoscope 14 of the catheter system 10 may be packaged together as a kit (e.g., a catheter system or installation kit). In one example, the catheter 12 may be pre-mounted on the insertion tube 18 of the endoscope 14 when packaged. This way, when a medical professional opens the package the catheter 12 is ready to be inserted into the body lumen using the endoscope 14, as described in more detail below. For example, in one embodiment, the catheter 12 may be molded on the endoscope 14. In one embodiment, the package may include a variety of different sizes (e.g., lengths) of catheters 12 so the medical professional can select the right size of catheter. After the medical professional selects the desired size of the catheter 12, the medical professional can then use the endoscope 14 to install the catheter.

In operation, the medical professional uses the catheter system 10 to place the catheter 12 in a body lumen of a patient. To place the catheter 12 in the body lumen, the catheter is mounted on the insertion tube 18 of the endoscope 14. In this arrangement, the insertion tube 18 of the endoscope 14 is disposed in the lumen 36 of the catheter 12. To mount the catheter 12 on the endoscope 14, the insertion tube 18 of the endoscope is positioned or inserted into the lumen 36 of the catheter. In one embodiment, the catheter 12 is not pre-mounted on the endoscope 14. In this case, the medical professional mounts the catheter 12 on the endoscope 14. If the catheter 12 is part of a kit with a variety of different sizes of catheters, the medical professional selects the desired size of catheter and mounts the selected catheter on the endoscope 14, as described above. In another embodiment, the catheter 12 comes to the medical professional already mounted on the endoscope 14 (e.g., the catheter system 10 is packaged or manufactured with the catheter mounted on the endoscope).

After the catheter 12 is mounted on the endoscope 14, the catheter system 10 is ready to be used. The medical professional inserts the catheter 12 into the body lumen of the patient using the endoscope 14. As the medical professional inserts the catheter 12 and endoscope 14, the medical professional may actuate the actuator 24 to guide the catheter 12 and endoscope 14 along the body lumen. After the catheter 12 is positioned in the body lumen, the inflatable balloon 40 is inflated to secure the catheter in the body. For example, once the distal end portion 34 of the elongate body 30 is positioned in the bladder of the patient, the inflatable balloon is inflated to hold the distal end portion 34 in the bladder. At any time while the endoscope 14 is in the body lumen, such as while the catheter 12 is being moved along the body lumen or when the distal end of the insertion tube is in the bladder, the medical professional can view the inside of the patient body (e.g., the body lumen or bladder) with the endoscope 14. The endoscope 14 looks or views through the transparent tip 38 of the elongate body 30. For example, the camera 20 of the endoscope 14 captures images and/or videos of the patient by looking through the transparent tip 38. The medical professional can use these images and/or video to visually inspect the body (e.g., body lumen, bladder) of the patient. After the catheter 12 is in the appropriate position in the lumen body and the balloon 40 is inflated, the insertion tube 18 of the endoscope 14 is withdrawn or removed from the lumen 36 of the catheter. The catheter 12 is now installed in the patient. It is understood these steps may be performed in other orders and that the method of operation may include fewer or additional steps than disclosed herein. It is also contemplated that some of the steps may be performed before, contemporaneously with, or after other steps in the operation.

Modifications and variations of the disclosed embodiments are possible without departing from the scope of the invention defined in the appended claims. For example, where specific dimensions are given, it will be understood that they are exemplary only and other dimensions are possible.

When introducing elements of the present invention or the one or more embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

As various changes could be made in the above apparatuses, systems, and methods without departing from the scope of the invention, it is intended that all matter contained in the above description and shown in the accompanying drawings shall be interpreted as illustrative and not in a limiting sense.

What is claimed is:

1. A catheter system comprising:
an endoscope having an insertion tube sized and shaped to be inserted into a body lumen of a patient; and
a catheter including an elongate body having an axis and proximal and distal end portions space apart from one another along the axis, the elongate body being sized and shaped to be received in the body lumen, the elongate body defining a lumen extending between the proximal and distal end portions, the lumen being sized and shaped to receive the insertion tube of the endoscope.

2. The catheter system of claim 1, wherein the lumen has a lumen diameter that is larger than an insertion tube diameter of the insertion tube of the endoscope.

3. The catheter system of claim 1, wherein the lumen diameter is about 5.4 mm.

4. The catheter system of claim 1, wherein the lumen has an open proximal end at the proximal end portion, the open proximal end sized and shaped to receive the insertion tube of the endoscope.

5. The catheter system of claim 1, wherein the elongate body includes a tip at the distal end portion, the tip defining a closed distal end of the lumen.

6. The catheter system of claim 5, wherein the tip is transparent.

7. The catheter system of claim 5, wherein the tip comprises a first material and a remainder of the elongate body comprises one or more additional materials that are different from the first material.

8. The catheter system of claim 1, wherein the elongate body is transparent.

9. The catheter system of claim 1, wherein the elongate body includes an inflatable balloon at the distal end portion.

10. The catheter system of claim 9, wherein the elongate body includes a tip at the distal end portion, the elongate body defining at least one fluid inlet in fluid communication with the lumen, the at least one fluid inlet being disposed between the tip and the inflatable balloon.

11. The catheter system of claim 10, wherein the at least one fluid inlet comprises a first fluid inlet and a second fluid inlet, the first and second fluid inlets disposed on generally opposite sides of the elongate body.

12. The catheter system of claim 1, wherein the catheter is a urinary catheter.

13. The catheter system of claim 1, wherein the insertion tube of the endoscope is disposed in the lumen of the catheter such that that as manufactured the catheter is mounted on the endoscope.

14. The catheter system of claim 1, wherein the elongate body includes a transparent tip at the distal end portion, and wherein the endoscope includes a camera arranged to look through the transparent tip of the elongate body.

15. A method of using a catheter, the method comprising:
inserting the catheter into a body lumen of a patient using an endoscope received in the catheter, the catheter comprising an elongate body having an axis and proximal and distal end portions space apart from one another along the axis, the elongate body defining a lumen extending between the proximal and distal end portions, wherein an insertion tube of the endoscope is disposed in the lumen.

16. The method of claim 15, further comprising mounting, before said inserting, the catheter on the insertion tube of the endoscope by positioning the insertion tube of the endoscope in the lumen of the catheter.

17. The method of claim 15, further comprising withdrawing, after said inserting, the insertion tube of the endoscope from the lumen of the catheter.

18. The method of claim 17, further comprising inflating, after said inserting, an inflatable balloon at the distal end portion of the elongate body.

19. The method of claim 15, wherein the elongate body includes a transparent tip at the distal end portion.

20. The method of claim 19, further comprising viewing, after said inserting, through the transparent tip a portion of the patient with the endoscope.
